(19) United States
(12) Patent Application Publication
(10) Pub. No.: US 2022/0323669 A1
(43) Pub. Date: Oct. 13, 2022

(54) MULTI-FUNCTION IRRIGATION-ASPIRATION TUBING FOR OCULAR SURGERY DEVICES

(71) Applicant: Bausch + Lomb Ireland Limited, Dublin (IE)

(72) Inventor: James T. Perkins, St. Charles, MO (US)

(73) Assignee: Bausch + Lomb Ireland Limited, Dublin (IE)

(21) Appl. No.: 17/229,114

(22) Filed: Apr. 13, 2021

(51) Int. Cl. A61M 3/02; A61F 9/007

(52) U.S. Cl. CPC A61M 3/0283; A61F 9/00745; A61M 3/0258; A61M 2205/12

(57) ABSTRACT

A device for ocular surgery includes a surgical handpiece including a power cord and an aspiration needle for removing ocular material from an eye, and co-extruded tubing. The tubing includes an aspiration tube for receiving the ocular material removed from the eye by the surgical handpiece, an irrigation tube integrally formed with the aspiration tube for supplying irrigation fluid to the surgical handpiece, and a ridge defined along at least a majority of a length of the co-extruded tubing, the ridge defining a groove for receiving the power cord of the surgical handpiece.

### FIELD

The present disclosure relates to multi-function irrigation-aspiration tubing for ocular surgery devices.

### BACKGROUND

This section provides background information related to the present disclosure which is not necessarily prior art.

A cataract clouds the natural lens of the eye, which includes mostly water and protein. Over time, these proteins may clump together to obscure the lens. This is generally corrected by removing the cataract lens and replacing it with a clear lens implant, such as through phacoemulsification eye surgery.

Phacoemulsification is a surgery technique on an eye where the internal lens is emulsified with a phacoemulsification (e.g., phaco) needle tip, which is driven to vibrate ultrasonically by an ultrasonic mechanism in the phaco surgical handpiece. The ultrasonic vibration of the phaco needle creates a significant temperature rise of the needle, which can occur essentially instantaneously. The emulsified lens material (which is mostly fluid) is aspirated from the eye through the phaco needle and replaced with an irrigation fluid (e.g., a balanced salt solution (BSS), etc.). Intraocular pressure (IOP) is maintained in the eye while the phaco needle is aspirating ocular material from the eye by continuously infusing saline solution into the eye. The constant replenishment of fluids in the eye is important to avoid collapse of the anterior chamber of the eye. The irrigation fluid also cools the heating effects of the vibrating phaco needle, thus preventing burning of eye tissue at the incision site. Occasionally, large chunks of ocular material clog the phaco needle, which interrupts the aspiration flow and in turn causes interruption in the irrigation flow.

### SUMMARY

This section provides a general summary of the disclosure, and is not a comprehensive disclosure of its full scope or all of its features.

According to one aspect of the present disclosure, a device for ocular surgery includes a surgical handpiece including a power cord and an aspiration needle for removing ocular material from an eye, and co-extruded tubing. The tubing includes an aspiration tube for receiving the ocular material removed from the eye by the surgical handpiece, an irrigation tube integrally formed with the aspiration tube for supplying irrigation fluid to the surgical handpiece, and a ridge defined along at least a majority of a length of the co-extruded tubing, the ridge defining a groove for receiving the power cord of the surgical handpiece.

According to another aspect of the present disclosure, tubing for an ocular surgical handpiece is disclosed. The tubing includes an aspiration tube for receiving ocular material removed from the eye by the surgical handpiece, and an irrigation tube integrally formed with the aspiration tube for supplying irrigation fluid to the surgical handpiece. A first ridge is defined along at least a majority of a length of the tubing, and the ridge defines a groove for receiving a power cord of the surgical handpiece. A second ridge is defined along at least a majority of the length of the co-extruded tubing.

Further aspects and areas of applicability will become apparent from the description provided herein. It should be understood that various aspects of this disclosure may be implemented individually or in combination with one or more other aspects. It should also be understood that the description and specific examples herein are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a block diagram of a device for ocular surgery, according to the prior art.

FIG. 2 is a sectional view of tubing of the ocular surgery device of FIG. 1, taken at A-A′.

Corresponding reference numerals indicate corresponding parts or features throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Disclosed herein are example embodiments of co-extruded tubing for ocular surgical procedures, such as phacoemulsification surgery, etc. The co-extruded tubing may connect a surgical handpiece to a console to control fluid flow to and from the surgical handpiece. The tubing may have any suitable length for performing ocular surgery with the handpiece, and may be up to six feet in length in some cases.

The co-extruded tubing may include two tubes separated by a wall, to separately allow fluid flow to and from the surgical handpiece. For example, an irrigation tube may deliver irrigation fluid from a source of balanced salt solution (BSS) to a surgical handpiece and to the eye during surgery. The delivery of BSS maintains the intra-ocular pressure (IOP) of the eye as tissue and fluid is aspirated from the eye through the surgical handpiece.

The other tube is an aspiration tube that connects the surgical handpiece to an aspiration reservoir (e.g., a bag, cassette, etc.) in the console that also houses the aspiration pump source. Preferably, the aspiration tubing is as non-compliant (e.g., not flexible, resists dilation, resists collapse, etc.) as possible.

The irrigation tube may be very compliant and easily expandable so that when a vacuum surge occurs (e.g., typically after a piece of cataract tissue occluding a phacoemulsification needle tip has cleared), there is sufficient irrigation fluid supply to prevent eye chamber collapse, which would be very harmful to the patient. In various embodiments, other suitable approaches for dealing with post-occlusion surge may be incorporated in the system.

As described further below, the co-extruded tubing may include a center ridge that runs at least a majority of the length of the tubing, and a groove for receiving a power cord for the phacoemulsification handpiece, a fragmentation handpiece, etc. This aids in cord management and may prevent or greatly reduce kinks in the tubes.

Some prior art techniques teach embedding wire in one or both tube walls, using cladding, or making the aspiration tube wall thicker than the irrigation tube wall to provide anti-kink benefits. In various aspects of the present disclosure described herein, including external ridge(s) along the co-extruded tubing (e.g., ribs on an external surface of the tubing, etc.), provides increased bend resistance and prevents kinking for co-extruded aspiration and irrigation tubing.

In addition, at least one of the external ridges may be configured to allow the electrical cable for the surgical handpiece to be inserted in a groove of the external ridge, therefore reducing the number of lines running through the operating area. This reduces the number of lines that need to be managed by the operating room staff during the surgery. The surgical handpiece ergonomics and reliability are improved because the aspiration and irrigation lines may act as a strain relief. This may also reduce issues at the interface of the handpiece and cable.

The co-extruded tubing may be manufactured by producing both the irrigation tube and the aspiration tube from the same material at the same time. Each of the aspiration and irrigation tubes may have a standard circular shape with a uniform wall thickness, although in other embodiments different opening shapes may be used, different wall thicknesses may be used, etc.

As the tubing is packaged, or during surgery if the user is not careful, the tubing can be pulled or placed in a position that may cause the tubing to be pinched or kinked, thus causing a reduction in fluid flow. The reduction in irrigation flow can be detrimental and cause complications (e.g., chamber shallowing, a broken capsule, etc.), during the surgery. Example embodiments described herein may reduce the likelihood of pinched or kinked tubing and resulting complications, via the one or more external ridges located on an exterior of the co-extruded tubing.

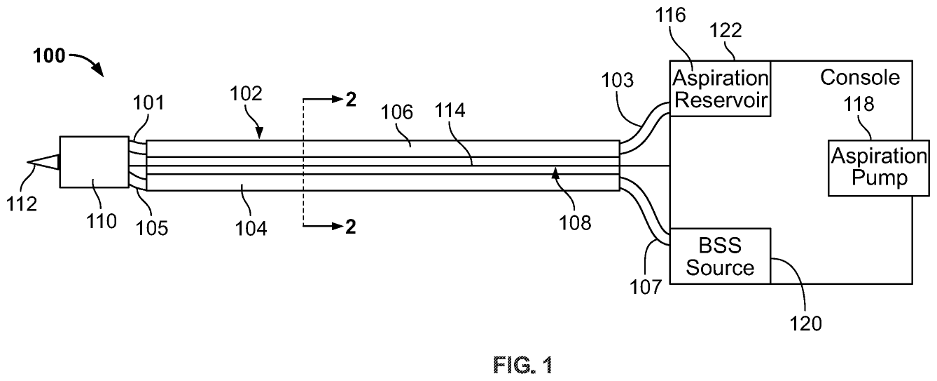

A device for ocular surgery according to one example embodiment of the present disclosure is illustrated in FIG. 1 and indicated generally by reference number 100. The ocular surgery device 100 includes a surgical handpiece 110 including a power cord 114 and an aspiration needle 112 for removing ocular material from an eye.

The device 100 also includes co-extruded tubing 102. The tubing 102 includes an aspiration tube 106 for receiving the ocular material removed from the eye by the surgical handpiece 110, and an irrigation tube 104 integrally formed with the aspiration tube 106 for supplying irrigation fluid to the surgical handpiece 110. A ridge is defined along at least a majority of a length of the co-extruded tubing 102. The ridge defines a groove 108 for receiving the power cord 114 of the surgical handpiece 110. Short tubing sections 101, 103, 105, and 107 may be provided to facilitate connection of the tubing to the handpiece 110 and the console 122.

As shown in FIG. 1, the device 100 optionally includes a console 122 including a source of balanced salt solution (BSS) 120. The irrigation tube 104 is adapted to supply the irrigation fluid from the source of BSS 120 to the surgical handpiece 110 during the ocular surgery, to maintain an inter-ocular pressure of the eye as the ocular material is aspirated from the eye through the surgical handpiece 110. The irrigation tube 104 may include a compliant material adapted to expand in response to a vacuum surge to supply sufficient irrigation fluid to inhibit eye chamber collapse. Although FIG. 1 illustrates a BSS source 120 located in the console 122, other embodiments may use other suitable irrigation fluids, may use irrigation sources from locations other than the console 122, etc.

The console 122 also includes an aspiration reservoir 116. The aspiration tube 106 is connected between the surgical handpiece 110 and the aspiration reservoir 116 (e.g., to supply fluid that has been aspirated from the eye from the surgical handpiece 110 to the aspiration reservoir). The aspiration tube 106 may include a non-compliant material adapted to inhibit flexion, dilation and collapse of the aspiration tube 106 during the ocular surgery. The aspiration reservoir 116 may include any suitable bag, cassette, etc. for receiving the fluid.

An aspiration pump 118 is also housed in the console 122, and may be used to generate a vacuum pressure for suctioning fluid through the aspiration tube 106 from the surgical handpiece 110 to the aspiration reservoir 116. Although FIG. 1 illustrates the aspiration reservoir 116 and the aspiration pump 118 located in the console 122, other embodiments may have the aspiration reservoir 116 and the aspiration pump 118 housed in other locations.

For example, during phacoemulsification surgery the internal lens of the eye is emulsified with the phacoemulsification needle 112, which is driven to vibrate ultrasonically by an ultrasonic mechanism in the surgical handpiece 110. The ultrasonic vibration of the needle 112 significantly increases the temperature of the needle 112. The emulsified lens material is aspirated from the eye through the needle 112 and the aspiration tube 106 to the aspiration reservoir 116 in the console 122, and replaced with an irrigation fluid from the BSS source 120 via the irrigation tube 104.

Intraocular pressure (IOP) is maintained in the eye while the needle 112 is aspirating ocular material from the eye by continuously infusing saline solution into the eye. The constant replenishment of fluids in the eye is important to avoid collapse of the anterior chamber of the eye. The irrigation fluid also cools the heating effects of the vibrating needle 112, thus preventing burning of eye tissue at the incision site. Occasionally, large chunks of ocular material clog the needle 112, which may interrupt the aspiration flow through the aspiration tube 106 and may cause an interruption in the irrigation flow through the irrigation tube 104.

In order to facilitate improved flow of fluid in the irrigation tube 104 and the aspiration tube 106, a ridge 108 is located along at least a majority of the co-extruded tubing 102. As shown in FIG. 1, the ridge 108 is disposed along an entire length of the co-extruded tubing 102. In other embodiments, the ridge 108 may be disposed along less than the entire length of the tubing 102, may be disposed along at least a majority (e.g., greater than half) of the length of the tubing 102, etc. The tubing 102 may have any suitable length for allowing a surgeon to move the handpiece 110 as needed during ocular surgery (e.g., up to six feet, etc.).

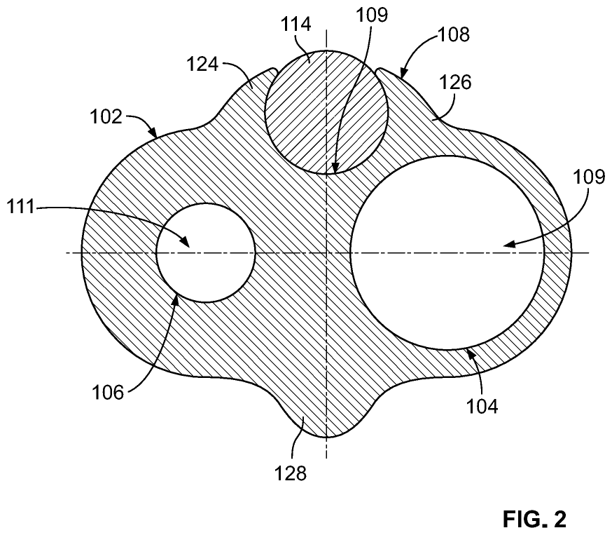

FIG. 2 illustrates a cross-sectional view of the co-extruded tubing 102 of FIG. 1, taken at A-A′. As shown in FIG. 2, the ridge 108 may be disposed along an external surface of the co-extruded tubing 102 to increase bend resistance of the co-extruded tubing 102. The external surface location of the ridge 108 may inhibit kinking of the co-extruded tubing 102 along both vertical and horizontal axes of the co-extruded tubing 102.

As shown in FIG. 2, the tubing 102 includes a second ridge 128 that is located on an external surface of the tubing 102 opposite the ridge 108. For example, the co-extruded tubing 102 may include multiple ridges that are each disposed along at least a majority of the length the tubing 102. Although FIG. 2 illustrates two ridges 108 and 128 located on opposite sides of the tubing 102, other embodiments may include more or less ridges, ridges positioned at other locations, etc.

The groove 109 of the ridge 108 includes two resilient arms 124 and 126. The resilient arms 124 and 126 are adapted to removably retain the power cord 114. For example, a user may insert the power cord 114 into the groove 109 by pressing the power cord 114 between the resilient arms 124 and 126, to hold the power cord 114 in place via a friction fit, a press fit, etc. The resilient arms 124 and 126 facilitate easy removal or positioning of the power cord 114 to fit different surgical setups.

In some embodiments, a diameter of the groove 109 may be about 0.095 inches. Although FIG. 2 illustrates the groove 109, in other embodiments the groove may have other suitable shapes for removably retaining the power cord 114. Similarly, other embodiments may have more or less than two resilient arms, resilient arms having different structural shapes, etc.

The irrigation tube 104 and the aspiration tube 106 each define a circular opening, and have different wall thicknesses. For example, in some embodiments a diameter of the opening 107 of the aspiration tube 106 is 1/16 of an inch, and a diameter of the opening 105 of the irrigation tube 104 is 5/32 of an inch.

As described above, the walls of the irrigation tube 104 and the walls of the aspiration tube 106 may include the same material. In other embodiments, the irrigation tube 104 and the aspiration tube may have other diameters, may have the same diameter as one another, may have the same wall thickness as one another, may have openings including shapes other than circular, etc.

According to another example embodiment of the present disclosure, tubing for an ocular surgical handpiece includes an aspiration tube for receiving ocular material removed from the eye by the surgical handpiece, and an irrigation tube integrally formed with the aspiration tube for supplying irrigation fluid to the surgical handpiece.

A first ridge is defined along at least a majority of a length of the tubing, with the ridge defining a groove for receiving a power cord of the surgical handpiece. A second ridge is defined along at least a majority of the length of the co-extruded tubing.

The first ridge and the second ridge may be disposed along opposite external sides of the tubing to increase bend resistance of the tubing and to inhibit kinking of the tubing along both vertical and horizontal axes of the tubing. The groove may include first and second resilient arms to removably retain the power cord of the ocular surgical handpiece.

As described herein, the surgical handpeice 110, the console 122, and any control circuits for the components of the device 100 may include a microprocessor, microcontroller, integrated circuit, digital signal processor, etc., which may include memory. The surgical handpeice 110, the console 122, and any control circuits may be configured to perform (e.g., operable to perform, etc.) any processes for controlling the device 100 using any suitable hardware and/or software implementation. For example, the surgical handpeice 110, the console 122, and any control circuits may execute computer-executable instructions stored in a memory, may include one or more logic gates, control circuitry, etc.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A device for ocular surgery, the device comprising:
a surgical handpiece including a power cord and an aspiration needle for removing ocular material from an eye; and
co-extruded tubing including:
an aspiration tube for receiving the ocular material removed from the eye by the surgical handpiece;
an irrigation tube integrally formed with the aspiration tube for supplying irrigation fluid to the surgical handpiece; and
a ridge defined along at least a majority of a length of the co-extruded tubing, the ridge defining a groove for receiving the power cord of the surgical handpiece.

2. The device of claim 1, further comprising a source of balanced salt solution (BSS), wherein the irrigation tube is adapted to supply the irrigation fluid from the source of BSS to the surgical handpiece during the ocular surgery to maintain an inter-ocular pressure of the eye as the ocular material is aspirated from the eye through the surgical handpiece.

3. The device of claim 2, further comprising an aspiration reservoir, wherein the aspiration tube is connected between the surgical handpiece and the aspiration reservoir.

4. The device of claim 3, further comprising a console and an aspiration pump housed in the console, wherein the aspiration reservoir comprises a bag or a cassette housed in the console.

5. The device of claim 1, wherein the ridge is disposed along an external surface of the co-extruded tubing to increase bend resistance of the co-extruded tubing and to inhibit kinking of the co-extruded tubing along both vertical and horizontal axes of the co-extruded tubing.

6. The device of claim 5, wherein the ridge is a first ridge, the co-extruded tubing further comprising a second ridge disposed along at least a majority of the length of the co-extruded tubing.

7. The device of claim 6, wherein the second ridge is located on a side of the co-extruded tubing opposite to a side on which the first ridge is located.

8. The device of claim 1, wherein the groove includes first and second resilient arms to removably retain the power cord.

9. The device of claim 8, wherein a diameter of the groove is 0.095 inches.

10. The device of claim 1, wherein the aspiration tube and the irrigation tube comprise the same tube material.

11. The device of claim 1, wherein the aspiration tube and the irrigation tube each define a circular opening and have a uniform wall thickness.

12. The device of claim 11, wherein:
a diameter of the opening of the aspiration tube is 1/16 of an inch; and/or
a diameter of the opening of the irrigation tube is 5/32 of an inch.

13. The device of claim 1, wherein the length of the co-extruded tubing is less than or equal to six feet.

14. The device of claim 1, wherein the ridge is disposed along an entire length of the co-extruded tubing.

15. The device of claim 1, wherein the surgical handpiece comprises a phacoemulsification handpiece or a fragmentation handpiece.

16. The device of claim 1, wherein the aspiration tube comprises a non-compliant material adapted to inhibit flexion, dilation and collapse of the aspiration tube during the ocular surgery.

17. The device of claim 1, wherein the irrigation tube comprises a compliant material adapted to expand in response to a vacuum surge to supply sufficient irrigation fluid to inhibit eye chamber collapse.

18. Tubing for an ocular surgical handpiece, the tubing comprising:
an aspiration tube for receiving ocular material removed from an eye by the surgical handpiece;
an irrigation tube integrally formed with the aspiration tube for supplying irrigation fluid to the surgical handpiece;
a first ridge defined along at least a majority of a length of the tubing, the first ridge defining a groove for receiving a power cord of the surgical handpiece; and
a second ridge defined along at least a majority of the length of the aspiration tube and the irrigation tube.

19. The tubing of claim 18, wherein the first ridge and the second ridge are disposed along opposite external sides of the tubing to increase bend resistance of the tubing and to inhibit kinking of the tubing along both vertical and horizontal axes of the tubing.

20. The tubing of claim 18, wherein:
the groove includes first and second resilient arms; and
the first and second resilient arms are adapted to removably retain the power cord of the ocular surgical handpiece.
